(19) United States
(12) Patent Application Publication
(10) Pub. No.: US 2023/0143454 A1
(43) Pub. Date: May 11, 2023

(54) COMPOSITIONS AND METHODS FOR EMERGENCY RESCUE

(71) Applicant: Purdue Pharma L.P., Stamford, CT (US)

(72) Inventors: Haiyong Hugh Huang, Princeton Junction, NJ (US); Manjunath S. Shet, White Plains, NY (US)

(21) Appl. No.: 17/918,012

(22) PCT Filed: Feb. 26, 2021
(86) PCT No.: PCT/US2021/019903

§ 371 Date: Oct. 10, 2022

Related U.S. Application Data
(60) Provisional application No. 63/018,062, filed on Apr. 30, 2020.

(51) Int. Cl. A61K 31/137; A61K 31/46; A61K 9/00; A61M 5/20; A61K 47/02

(52) U.S. Cl. CPC A61K 31/137; A61K 31/46; A61K 9/0019; A61M 5/20; A61K 47/02

(57) ABSTRACT

Disclosed in certain embodiments is a parenteral formulation comprising a therapeutically effective amount of an emergency rescue active pharmaceutical ingredient and a parenterally acceptable adjuvant in an amount that enhances the absorption of the emergency rescue active pharmaceutical ingredient (ER-API) into the systemic circulation. The enhanced absorption may be evidence by a faster onset time, a faster Tₘₐₓ and/or a greater Cₘₐₓ and/or a greater AUCₗₐₛₜ and/or a greater AUC_{0.25} and/or a greater AUC_{0.5} and/or a greater AUC_{0.75} and/or a greater AUC₁ and/or increased bioavailability as compared to formulations without the adjuvant.

### FIELD OF THE INVENTION

The present invention relates to the field of pharmaceutical compositions for providing emergency rescue to a subject from an allergic reaction (e.g., anaphylactic shock) or from poisoning, pre-dosing a subject as protection against an allergic reaction, methods of providing emergency rescue, and drug delivery systems thereof.

### BACKGROUND OF THE INVENTION

Reactions to certain allergens and/or poisons could sometimes be very severe and even fatal. Subjects experiencing symptoms associated with allergic reactions would benefit from pharmaceutical products that could rapidly counteract, treat, prevent, and/or reduce such symptoms. Similarly, subjects exposed to poisonous substances unintentionally or subjects that are at a risk of being potentially exposed to poisonous substances would benefit from pharmaceutical products that could provide an antidote to the poison, counteract, treat, prevent, and/or reduce symptoms associated with poisoning.

Allergy symptoms and poisoning symptoms include, without limitations, anaphylactic shock, difficulty breathing, abdominal cramping or pain, pain or tightness in the chest, diarrhea, difficulty swallowing, dizziness (vertigo), anxiety, fear, flushing of the face, nausea or vomiting, heart palpitations, swelling of the airways and/or face and/or eyes and/or tongue, unconsciousness, weakness, hives, itching, congestion, rash, scratchy throat, watery or itchy eyes, drop in blood pressure, temporary blockade of severe or life threatening muscarinic effects, bardyasystolic cardiac arrest, and other symptoms suggestive of an allergic reaction and/or poisoning.

To counteract symptoms of allergic reactions and/or poisoning, emergency personnel or others may administer an antidote such as an intramuscular injection of an emergency rescue active pharmaceutical ingredient. Given that the administered antidote needs to be absorbed into the bloodstream, there inevitably will be a lag time from the time of antidote administration to the time that the antidote reaches therapeutic levels sufficient to effectively counteract the effects of the allergen or poison. Unfortunately, this lag time can result in the antidote treatment not being effective enough in sufficient time to prevent morbidity and mortality due to the allergic reaction and poisoning effect.

There exists a need in the art for a pharmaceutical composition for rescuing a subject from allergic reactions and/or poisoning, and a method of rescuing a subject from allergic reactions and/or poisoning through which enhanced absorption is achieved.

### OBJECTS AND SUMMARY OF THE INVENTION

It is an object of certain embodiments of the present invention to provide a pharmaceutical composition (e.g., a parenteral formulation or an intranasal formulation) for rescuing a subject from an allergic reaction (e.g., anaphylactic shock) or from poisoning, or for preventing (or reducing the risk in) a subject from experiencing an allergic reaction (e.g., anaphylactic shock).

It is an object of certain embodiments of the present invention to provide a parenteral formulation for rescuing a subject from an allergic reaction (e.g., anaphylactic shock) or from poisoning, or for preventing (or reducing the risk in) a subject from experiencing an allergic reaction (e.g., anaphylactic shock) or from poisoning. In one embodiment, the parenteral formulation is suitable for intramuscular administration. In another embodiment, the parenteral formulation is suitable for subcutaneous administration.

It is an object of certain embodiments of the present invention to provide an intranasal formulation for rescuing a subject from an allergic reaction (e.g., anaphylactic shock) or from poisoning, or for preventing (or reducing the risk in) a subject from experiencing an allergic reaction (e.g., anaphylactic shock) or from poisoning.

It is an object of certain embodiments of the present invention to provide a method for rescuing a subject from an allergic reaction (e.g., anaphylactic shock) or from poisoning, or for preventing (or reducing the risk in) a subject from experiencing an allergic reaction (e.g., anaphylactic shock) or from poisoning.

It is an object of certain embodiments of the present invention to provide a method of prophylactically administering a pharmaceutical composition as disclosed herein to a subject who is at risk of being exposed to a substance (e.g., pollen, nuts such as peanuts and tree nuts, milk, eggs, shellfish, wheat, soy, fish, and the like) to which the subject is allergic, or to a poisonous substance (e.g., nerve agent poisoning, insecticide poisoning, and the like).

It is an object of certain embodiments of the present invention to provide a drug delivery system for rescuing a subject from an allergic reaction or from poisoning, or from preventing (or reducing the risk in) a subject from experiencing an allergic reaction (e.g., an anaphylactic shock) or poisoning.

The above objects and others may be achieved by the present invention which in certain embodiments is directed to a pharmaceutical composition for providing emergency rescue to a subject experiencing an allergic reaction or poisoning, or for preventing (or reducing the risk of) an allergic reaction poisoning in a subject. In certain embodiments, the pharmaceutical composition comprises a therapeutically effective amount of an emergency rescue active pharmaceutical ingredient (ER-API), and a parenterally acceptable absorption enhancing amount of an adjuvant that promotes, enhances, or quickens the systemic absorption rate of the ER-API upon intramuscular or subcutaneous injection, wherein the ER-API is not an opioid antagonist. The adjuvant may be selected from appropriate absorption-enhancing agents currently known or those that would be readily appreciated by an ordinary skilled artisan (in formulation and medical fields) for such use. In certain non-limiting embodiments, the adjuvant is selected from the group of nitric oxide inducers, niacin, niacin derivatives, niacin metabolites, phosphodiesterase inhibitors, angiotensin converting enzyme (ACE) inhibitors, angiotensin receptor blockers, calcium channel blockers, nitrates or combinations thereof. In one embodiment, the adjuvant is magnesium chloride.

In certain embodiments, the ER-API is epinephrine, or atropine, or a pharmaceutically acceptable salt thereof, or a combination thereof.

In certain embodiments, the pharmaceutical composition of the present invention comprises a therapeutically effective amount of an ER-API and an absorption-enhancing effective amount of a pharmaceutically acceptable adjuvant (e.g., parenterally acceptable adjuvant or an intranasally acceptable adjuvant), wherein the composition provides a time to onset of action of the ER-API of 5 minutes or less post intramuscular or subcutaneous injection or upon intranasal or inhalative administration to a subject experiencing, or at risk of experiencing an allergic reaction.

In certain embodiments, the composition provides a mean time to maximum plasma concentration of ER-API of about 2.0 hours or less post intramuscular injection to a population of healthy subjects, or about 1.0 hour or less post subcutaneous injection to a population of healthy subjects.

In certain embodiments, the present invention is directed to a method of providing emergency rescue or allergic reaction prevention to a subject experiencing, or at risk of experiencing, an allergic reaction, comprising intramuscularly, subcutaneously, intranasally, or via inhalation administering to the subject a pharmaceutical composition as disclosed herein.

In certain embodiments, the present invention is directed to a drug delivery system comprising a device (e.g., an injection device) containing a pharmaceutical composition as disclosed herein. In one embodiment, such a device is suitable for delivering the pharmaceutical composition through injection. In certain embodiments, the composition is disposed within a pre-filled syringe, a vial, an injection pen, or an autoinjector. In one embodiment, such a device is suitable for delivering the pharmaceutical composition intranasally or via inhalation.

### BRIEF DESCRIPTION OF THE DRAWINGS

The above and other features of the present invention, their nature, and various advantages will become more apparent post consideration of the following detailed description, taken in conjunction with the accompanying drawings, in which:

FIG. 1 depicts the comparative mean plasma concentration profiles of a 0.2 mg dose of epinephrine with different levels of MgCl₂ adjuvant (0% (Group 1), 1% (w/v) (Group 2), and 2% (w/v) (Group 3)) after intramuscular administration in three dog subjects. For dog subjects receiving 0.2 mg dose of epinephrine with 0% MgCl₂, 0.9% (w/v) NaCl (in Group 1) was employed.

FIG. 2 depicts the comparative mean plasma concentration profiles of a 0.2 mg dose of atropine with different levels of MgCl₂ adjuvant (0% (Group 1), 1% (w/v) (Group 2), and 2% (w/v) (Group 3)) after intramuscular administration in three dog subjects. For dog subjects receiving 0.2 mg dose of atropine with 0% MgCl₂, 0.9% (w/v) NaCl (in Group 1) was employed.

### DEFINITIONS

As used herein, the singular forms “a,” “an,” and “the” include plural references unless the context clearly indicates otherwise. Thus, for example, reference to “an active agent” includes a single active agent as well as a mixture of two or more different active agents; and reference to an “excipient” includes a single excipient as well as a mixture of two or more different excipients, and the like.

As used herein, the term “about” in connection with a measured quantity or time, refers to the normal variations in that measured quantity or time, as expected by one of ordinary skill in the art in making the measurement and exercising a level of care commensurate with the objective of measurement. In certain embodiments, the term “about” includes the recited number ±10%, such that “about 10” would include from 9 to 11, or 1 hour would include from 54 minutes to 66 minutes.

As used herein, the term “active agent” refers to any material that is intended to produce a therapeutic, prophylactic, or other intended effect, whether or not approved by a government agency for that purpose. This term with respect to a specific agent includes the pharmaceutically active agent, and all pharmaceutically acceptable salts, solvates and crystalline forms thereof, where the salts, solvates and crystalline forms are therapeutically active.

As used herein, the terms “therapeutically effective” and an “effective amount” refer to the amount of active agent or the rate at which it is administered which is needed to produce a desired therapeutic result.

The term “subject” refers to a human or animal, who has presented a clinical manifestation of an allergic reaction suggesting the need for rescue treatment, or who is at risk of being exposed to a allergenic substance or to poison, who is treated prophylactically with an ER-API. The term “subject” may include a person or animal (e.g., dog) who is a patient being appropriately treated by a medical caregiver with an ER-API to treat, prevent, or reduce the risk of an allergic reaction or poisoning. The term “subject” may also include any person who appears to a not-clinically trained bystander to be experiencing an allergic reaction such as anaphylactic shock, difficulty breathing, abdominal cramping or pain, pain or tightness in the chest, diarrhea, difficulty swallowing, dizziness (vertigo), anxiety, fear, flushing of the face, nausea or vomiting, heart palpitations, swelling of the airways and/or face and/or eyes and/or tongue, unconsciousness, weakness, hives, itching, congestion, rash, scratchy throat, watery or itchy eyes, drop in blood pressure, and other symptoms suggestive of an allergic reaction. The term “subject” may also include any person who appears to a not-clinically trained bystander to be experiencing a poisoning reaction such as, temporary blockade of severe or life threatening muscarinic effects, bardyasystolic cardiac arrest, and other symptoms suggestive of poisoning.

The terms “treatment of” and “treating” include the administration of an active agent(s) with the intent to lessen the severity of a condition.

The terms “prevention of” and “preventing” include the avoidance of the onset of a condition by prophylactic administration of the active agent.

The term “condition” or “conditions” refers to those medical conditions which are commonly recognized as the result of an allergic reaction or poisoning, such as anaphylactic shock, difficulty breathing, abdominal cramping or pain, pain or tightness in the chest, diarrhea, difficulty swallowing, dizziness (vertigo), anxiety, fear, flushing of the face, nausea or vomiting, heart palpitations, swelling of the airways and/or face and/or eyes and/or tongue, unconsciousness, weakness, hives, itching, congestion, rash, scratchy throat, watery or itchy eyes, drop in blood pressure, temporary blockade of severe or life threatening muscarinic effects, bardyasystolic cardiac arrest, or a combination thereof, which can be treated, mitigated or prevented by timely administration to a subject of an effective amount of an ER-API.

The term “adjuvant” refers to an agent that is incorporated into a pharmaceutical composition to enhance the absorption of an active agent, e.g., by increasing Cₘₐₓ, shortening Tₘₐₓ, increasing one or more of AUCₗₐₛₜ, AUC_{0.25}, AUC_{0.5}, AUC_{0.75}, AUC₁, shortening onset time, or increasing bioavailability, or any combination thereof. An adjuvant may be inactive in all other respects or may provide an intended or unintended pharmacological effect in addition to enhancing the absorption of an active agent.

The term “enhanced absorption” refers to increase in Cₘₐₓ and/or shortened Tₘₐₓ and/or shortened onset time and/or increased AUCₗₐₛₜ and/or increases AUC_{0.25} and/or increased AUC_{0.5} and/or increased AUC_{0.75} and/or increased AUC₁ and/or increased bioavailability, or any combination thereof, of a formulation that includes an adjuvant as compared to the same formulation without an adjuvant.

The term “parenteral” means administration by injection, implantation or infiltration by a route such as intravenous, intraarterial, intracardiac, intraspinal, intraosseous, intraarticular, intrasynovial, intracutaneous, subcutaneous or intramuscular.

The term “injection” means administration to a discrete site through the skin or into tissue of a human or animal.

The term “implantation” means administration to a discrete site by embedding pharmaceutical composition into the skin, tissue, muscles, tendons, joints, or other body parts of a human or animal.

The term “infiltration” means administration into a discrete injection site, or surgical site or open wound.

T_{1/2} is a pharmacokinetic term referring to the time taken for half the initial dose of active agent administered to be eliminated from the body of the subject.

Tₘₐₓ is a pharmacokinetic term referring to the time at which Cmax is observed.

Cₘₐₓ is a pharmacokinetic term referring to the maximum plasma concentration of the active agent.

MRTₗₐₛₜ is a pharmacokinetic term referring to the mean residence time, calculated to the last observable time point (e.g., MRT₂ refers to the mean residence time, calculated to two hours).

AUCₗₐₛₜ is a pharmacokinetic term referring to the area under the plasma concentration as a function of time curve, calculated to the last observable time point (e.g., AUC₂ refers to the area under the curve, calculated to two hours).

AUC_{∞} is a pharmacokinetic term referring to the area under the plasma concentration as a function of time curve, extrapolated to infinity.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

### DETAILED DESCRIPTION

### Dosage Forms and Pharmaceutical Compositions

According to various embodiments, the present invention is related to a pharmaceutical composition for emergency rescue from an allergic reaction or poisoning. In certain embodiments, the invention is directed to a pharmaceutical composition comprising a therapeutically effective amount of an ER-API and a parenterally acceptable adjuvant (e.g., a pharmaceutically acceptable adjuvant) that promotes the absorption rate of the ER-API upon intramuscular or subcutaneous injection. Suitable adjuvants for the invention may comprise nitric oxide inducers, niacin, niacin derivatives, niacin metabolites, local anesthetics, phosphodiesterase inhibitors, angiotensin converting enzyme (ACE) inhibitors, angiotensin receptor blockers, calcium channel blockers, nitrates and combinations thereof. In certain embodiments, such a pharmaceutical composition provides for a quicker onset of action of the ER-API as compared to the same pharmaceutical composition but without the adjuvant.

The ER-API can be any emergency rescue active pharmaceutical ingredient currently known or those that would be readily appreciated by an ordinary skilled artisan (in formulation and medical fields) for such use that effectively counteracts or prevents and allergic reaction or poisoning. In certain embodiments, suitable ER-APIs comprise epinephrine, atropine, pharmaceutically acceptable salts thereof, or combinations thereof. In certain embodiments, the ER-API is epinephrine or a pharmaceutically acceptable salt thereof. In certain embodiments, the ER-API is atropine or a pharmaceutically acceptable salt thereof.

In certain embodiments, the invention is directed to a pharmaceutical composition comprising a therapeutically effective amount of an ER-API and a parenterally acceptable adjuvant, wherein the formulation provides a time to onset of action (i.e., the first detectable therapeutic effect associated with ER-API, e.g., detectable lessening or reduction of any of the symptoms associated with an allergic reaction or poisoning) of less than about 5 minutes post intramuscular or subcutaneous injection to a subject experiencing an allergic reaction or poisoning, or pretreating against potential allergen or poison exposure.

In certain embodiments, the pharmaceutical composition provides a time to onset of action (i.e., counteracting at least one symptom of an allergic reaction or poisoning) of about 4 minutes or less, about 3 minutes or less, about 2 minutes or less or about 1 minute or less post intramuscular or subcutaneous injection to a subject experiencing an allergic reaction or poisoning or needing pretreatment due to potential allergen or poison exposure. In certain embodiments, the pharmaceutical composition provides a time to onset of action (i.e., counteracting at least one symptom of an allergic reaction or poisoning) from greater than about 5 seconds, greater than about 10 seconds, greater than about 15 seconds, greater than about 30 seconds, greater than about 45 seconds or greater than about 1 minute to about 2 minutes or less, about 3 minutes or less, about 4 minutes or less, or about 5 minutes or less, or any sub-range therein, upon intramuscular or subcutaneous injection to a subject experiencing an allergic reaction or poisoning or to a subject needing pretreatment against potential allergen or poison exposure.

In other embodiments, the invention is directed to a pharmaceutical composition comprising a therapeutically effective amount of an ER-API and a parenterally acceptable adjuvant, wherein the formulation provides a mean time to achieve maximum plasma concentration of ER-API of about 2.0 hours or less post intramuscular injection to a population of subjects (e.g., healthy or otherwise healthy subjects) or about 1 hour or less post subcutaneous injection to a population of subjects (e.g., healthy or otherwise healthy subjects).

In other embodiments, the invention is directed to a pharmaceutical composition comprising a therapeutically effective amount of an ER-API and a parenterally acceptable adjuvant, wherein the formulation provides a mean time to achieve maximum plasma concentration of ER-API of about 2 hours or less, about 1.5 hours or less, about 1 hour or less, about 0.75 hour or less, about 0.5 hour or less, about 20 minutes or less, about 15 minutes or less, about 10 minutes or less, about 5 minutes or less, about 3 minutes or less, or about 1 minute or less post intramuscular injection to a population of subjects (e.g., healthy or otherwise healthy subjects). In other embodiments, the formulation provides a mean time to achieve maximum plasma concentration of ER-API from about 0.1 hour or more, about 0.2 hour or more, about 0.3 hour or more, or about 0.4 hour or more to any of about 0.5 hour or less, about 1 hour or less, about 1.5 hours or less, or about 2.0 hours or less post intramuscular injection to a population of subjects (e.g., healthy or otherwise healthy subjects).

In other embodiments, the invention is directed to a pharmaceutical composition comprising a therapeutically effective amount of an ER-API and a parenterally acceptable adjuvant, wherein the formulation provides a mean time to achieve maximum plasma concentration of ER-API of about 2 hours or less, about 1.5 hours or less, about 1 hour or less, about 0.9 hour or less, about 0.8 hour or less, about 0.75 hours or less, about 0.7 hour or less, about 0.6 hour or less, about 0.5 hour or less, about 20 minutes or less, about 15 minutes or less, about 10 minutes or less, about 5 minutes or less, about 3 minutes or less, or about 1 minute or less post subcutaneous injection to a population of subjects (e.g., healthy or otherwise healthy subjects). In other embodiments, the formulation provides a mean time to maximum plasma concentration of ER-API from about 0.1 hour or more, about 0.2 hour or more, about 0.3 hour or more, or about 0.4 hour or more to about 1.0 hour or about 0.5 hours or less post subcutaneous injection to a population of subjects (e.g., healthy or otherwise healthy subjects).

In certain embodiments, the composition provides a mean time to maximum plasma concentration of an ER-API of about 2.0 hours or less, about 1.5 hours or less, about 1 hour or less, about 0.9 hour or less, about 0.8 hours or less, about 0.75 hours or less, about 0.7 hour or less, about 0.6 hours or less, about 0.5 hours or less, about 20 minutes or less, about 15 minutes or less, about 10 minutes or less, about 5 minutes or less, about 3 minutes or less, or about 1 minute or less post intramuscular or subcutaneous injection to a population of subjects (e.g., healthy or otherwise healthy subjects) and also provides an onset of therapeutic action of less than 5 minutes, about 4 minutes or less, about 3 minutes or less, about 2 minutes or less or about 1 minute or less post intramuscular or subcutaneous injection to a subject experiencing an allergic reaction or poisoning or needing pretreatment due to potential allergen or poison exposure.

In certain embodiments, the invention is directed to a pharmaceutical composition comprising a therapeutically effective amount of epinephrine or a pharmaceutically acceptable salt thereof and a parenterally acceptable adjuvant, wherein the formulation provides a mean time to maximum plasma concentration of epinephrine (Tₘₐₓ) of about 2.0 hours or less, about 1.5 hours or less, about 1 hour or less, about 0.9 hour or less, about 0.8 hours or less, about 0.75 hours or less, about 0.7 hours or less, about 0.6 hours or less, about 0.5 hours or less, about 20 minutes or less, about 15 minutes or less, about 10 minutes or less, about 5 minutes or less, about 3 minutes or less, or about 1 minute or less post intramuscular or subcutaneous injection to a population of subjects (e.g., healthy or otherwise healthy subjects).

In certain embodiments, the invention is directed to a pharmaceutical composition comprising a therapeutically effective amount of epinephrine or a pharmaceutically acceptable salt thereof and a parenterally acceptable adjuvant, wherein the formulation provides a mean time to maximum plasma concentration of epinephrine (Tₘₐₓ) that is shorter than the mean time to maximum plasma concentration of epinephrine of a comparative formulation without an adjuvant, post intramuscular or subcutaneous injection to a population of subjects (e.g., healthy or otherwise healthy subjects). For instance, the mean Tₘₐₓ of the present invention may be about 1.1 times shorter, about 1.2 times shorter, about 1.3 times shorter, about 1.4 times shorter, about 1.5 times shorter, about 1.6 times shorter, about 1.7 times shorter, about 1.8 times shorter, about 1.9 times shorter, or about 2 times shorter than that of a comparative formulation without an adjuvant.

In certain embodiments, the invention is directed to a pharmaceutical composition comprising a therapeutically effective amount of epinephrine or a pharmaceutically acceptable salt thereof and a parenterally acceptable adjuvant, wherein the formulation provides a mean maximum plasma concentration of epinephrine (Cₘₐₓ) of at least about 15 ng/mL per 0.3 mg epinephrine or pharmaceutically acceptable salt thereof, post intramuscular or subcutaneous administration to a population of subjects (e.g., healthy or otherwise healthy subjects).

In certain embodiments, the formulation provides a mean maximum concentration of epinephrine of at least about 1 ng/mL, at least about 3 ng/mL, at least about 5 ng/mL, at least about 7 ng/mL, at least about 10 ng/mL, at least about 12 ng/mL, at least about 15 ng/mL, at least about 18 ng/mL, or at least about 20 ng/mL per 0.3 mg epinephrine or pharmaceutically acceptable salt thereof, post intramuscular or subcutaneous administration to a population of subjects (e.g., healthy or otherwise healthy subjects).

In certain embodiments, the formulation provides a mean maximum concentration of epinephrine of about 50 ng/mL or less, about 45 ng/mL or less, about 40 ng/mL or less, about 35 ng/mL or less, about 30 ng/mL or less, about 28 ng/mL or less, about 26 ng/mL or less, about 24 ng/mL or less, or about 22 ng/mL or less per 0.3 mg epinephrine or pharmaceutically acceptable salt thereof, post intramuscular or subcutaneous administration to a population of subjects (e.g., healthy or otherwise healthy subjects).

In certain embodiments, the invention is directed to a pharmaceutical composition comprising a therapeutically effective amount of epinephrine or a pharmaceutically acceptable salt thereof and a parenterally acceptable adjuvant, wherein the formulation provides a mean maximum plasma concentration of epinephrine (Cₘₐₓ) that is greater than the mean maximum plasma concentration of epinephrine of a comparative formulation without an adjuvant, post intramuscular or subcutaneous injection to a population of subjects (e.g., healthy or otherwise healthy subjects). For instance, Cₘₐₓ may be about 1.1 times greater, about 1.2 times greater, about 1.3 times greater, about 1.4 times greater, about 1.5 times greater, about 1.6 times greater, about 1.7 times greater, about 1.8 times greater, about 1.9 times greater, or about 2 times greater than that of a comparative formulation without an adjuvant.

In certain embodiments, the invention is directed to a pharmaceutical composition comprising a therapeutically effective amount of epinephrine or a pharmaceutically acceptable salt thereof and a parenterally acceptable adjuvant, wherein the formulation provides an AUCₗₐₛₜ that is greater or lower than the AUCₗₐₛₜ of epinephrine of a comparative formulation without an adjuvant, post intramuscular or subcutaneous injection to a population of subjects (e.g., healthy or otherwise healthy subjects). For instance, AUCₗₐₛₜ may be about 1.1 times greater or lower, about 1.2 times greater or lower, about 1.3 times greater or lower, about 1.4 times greater or lower, about 1.5 times greater or lower, about 1.6 times greater or lower, about 1.7 times greater or lower, about 1.8 times greater or lower, about 1.9 times greater or lower, or about 2 times greater or lower than that of a comparative formulation without an adjuvant.

In certain embodiments, the invention is directed to a pharmaceutical composition comprising a therapeutically effective amount of epinephrine or a pharmaceutically acceptable salt thereof and a parenterally acceptable adjuvant, wherein the formulation provides an AUC_{0.25}, AUC_{0.5}, AUC_{0.75}, or AUC₁ that is greater than the respective AUC_{0.25}, AUC_{0.5}, AUC_{0.75}, or AUC₁ of epinephrine of a comparative formulation without an adjuvant, post intramuscular or subcutaneous injection to a population of subjects (e.g., healthy or otherwise healthy subjects). For instance, AUC_{0.25}, AUC_{0.5}, AUC_{0.75}, or AUC₁ may be about 1.1 times greater, about 1.2 times greater, about 1.3 times greater, about 1.4 times greater, about 1.5 times greater, about 1.6 times greater, about 1.7 times greater, about 1.8 times greater, about 1.9 times greater, or about 2 times greater than that of a respective AUC_{0.25}, AUC_{0.5}, AUC_{0.75}, or AUC₁ of a comparative formulation without an adjuvant.

In certain embodiments, the invention is directed to a pharmaceutical composition comprising a therapeutically effective amount of atropine or a pharmaceutically acceptable salt thereof and a parenterally acceptable adjuvant, wherein the formulation provides a mean time to maximum plasma concentration of atropine (Tₘₐₓ) of about 3.0 hours or less, about 2.5 hours or less, about 2.0 hours or less, about 1.5 hours or less, about 1 hour or less, about 0.9 hour or less, about 0.8 hours or less, about 0.75 hours or less, about 0.7 hour or less, about 0.6 hours or less, about 0.5 hours or less, about 20 minutes or less, about 15 minutes or less, or about 10 minutes or less, about 5 minutes or less, about 3 minutes or less, or about 1 minute or less post intramuscular or subcutaneous injection to a population of subjects (e.g., healthy or otherwise healthy subjects).

In certain embodiments, the invention is directed to a pharmaceutical composition comprising a therapeutically effective amount of atropine or a pharmaceutically acceptable salt thereof and a parenterally acceptable adjuvant, wherein the formulation provides a mean time to maximum plasma concentration of atropine (Tₘₐₓ) that is shorter than the mean time to maximum plasma concentration of atropine of a comparative formulation without an adjuvant, post intramuscular or subcutaneous injection to a population of subjects (e.g., healthy or otherwise healthy subjects). For instance, the mean Tₘₐₓ of the present invention may be about 1.1 times shorter, about 1.2 times shorter, about 1.3 times shorter, about 1.4 times shorter, about 1.5 times shorter, about 1.6 times shorter, about 1.7 times shorter, about 1.8 times shorter, about 1.9 times shorter, or about 2 times shorter than that of a comparative formulation without an adjuvant.

In certain embodiments, the invention is directed to a pharmaceutical composition comprising a therapeutically effective amount of atropine or a pharmaceutically acceptable salt thereof and a parenterally acceptable adjuvant, wherein the formulation provides a mean maximum plasma concentration of atropine (Cₘₐₓ) of at least about 12 ng/mL per 2 mg atropine or pharmaceutically acceptable salt thereof, post intramuscular or subcutaneous administration to a population of subjects (e.g., healthy or otherwise healthy subjects).

In certain embodiments, the formulation provides a mean maximum concentration of atropine of at least about 12 ng/mL, at least about 15 ng/mL, at least about 20 ng/mL, at least about 25 ng/mL, or at least about 30 ng/mL per 2 mg atropine or pharmaceutically acceptable salt thereof, post intramuscular or subcutaneous administration to a population of subjects (e.g., healthy or otherwise healthy subjects).

In certain embodiments, the formulation provides a mean maximum concentration of atropine of about 100 ng/mL or less, about 75 ng/mL or less, about 50 ng/mL or less, about 45 ng/mL or less, about 40 ng/mL or less, about 35 ng/mL or less, about 30 ng/mL or less, about 25 ng/mL or less, or about 20 ng/mL or less per 2 mg atropine or pharmaceutically acceptable salt thereof, post intramuscular or subcutaneous administration to a population of subjects (e.g., healthy or otherwise healthy subjects).

In certain embodiments, the invention is directed to a pharmaceutical composition comprising a therapeutically effective amount of atropine or a pharmaceutically acceptable salt thereof and a parenterally acceptable adjuvant, wherein the formulation provides a mean maximum plasma concentration of atropine (Cₘₐₓ) that is greater than the mean maximum plasma concentration of atropine or a comparative formulation without an adjuvant, post intramuscular or subcutaneous injection to a population of subjects (e.g., healthy or otherwise healthy subjects). For instance, Cₘₐₓ may be about 1.1 times greater, about 1.2 times greater, about 1.3 times greater, about 1.4 times greater, about 1.5 times greater, about 1.6 times greater, about 1.7 times greater, about 1.8 times greater, about 1.9 times greater, or about 2 times greater than that of a comparative formulation without an adjuvant.

In certain embodiments, the invention is directed to a pharmaceutical composition comprising a therapeutically effective amount of atropine or a pharmaceutically acceptable salt thereof and a parenterally acceptable adjuvant, wherein the formulation provides an AUCₗₐₛₜ that is greater or lower than the AUCₗₐₛₜ of atropine or a comparative formulation without an adjuvant, post intramuscular or subcutaneous injection to a population of subjects (e.g., healthy or otherwise healthy subjects). For instance, AUCₗₐₛₜ may be about 1.1 times greater or lower, about 1.2 times greater or lower, about 1.3 times greater or lower, about 1.4 times greater or lower, about 1.5 times greater or lower, about 1.6 times greater or lower, about 1.7 times greater or lower, about 1.8 times greater or lower, about 1.9 times greater or lower, or about 2 times greater or lower than that of a comparative formulation without an adjuvant.

In certain embodiments, the invention is directed to a pharmaceutical composition comprising a therapeutically effective amount of atropine or a pharmaceutically acceptable salt thereof and a parenterally acceptable adjuvant, wherein the formulation provides an AUC_{0.25}, AUC_{0.5}, AUC_{0.75}, or AUC₁ that is greater than the respective AUC_{0.25}, AUC_{0.5}, AUC_{0.7}, or AUC₁ of atropine or a comparative formulation without an adjuvant, post intramuscular or subcutaneous injection to a population of subjects (e.g., healthy or otherwise healthy subjects). For instance, AUC_{0.25}, AUC_{0.5}, AUC_{0.75}, or AUC₁ may be about 1.1 times greater, about 1.2 times greater, about 1.3 times greater, about 1.4 times greater, about 1.5 times greater, about 1.6 times greater, about 1.7 times greater, about 1.8 times greater, about 1.9 times greater, or about 2 times greater than the respective AUC_{0.25}, AUC_{0.5}, AUC_{0.75}, or AUC₁ of a comparative formulation without an adjuvant.

In certain embodiments, the pharmacokinetic values described herein may be obtained from an individual subject (healthy or in therapeutic need thereof) or from a plurality of subjects (healthy or in therapeutic need thereof) post a parenteral administration of any of the pharmaceutical compositions disclosed herein.

The role of the adjuvant is to promote or quicken the systemic absorption rate of the ER-API (e.g., epinephrine or pharmaceutically acceptable salt thereof or atropine or pharmaceutically acceptable salt thereof) post intramuscular or subcutaneous injection. In certain embodiments, the adjuvant is a vasodilator. The vasodilator may be an angiotensin converting enzyme (ACE) inhibitor, an angiotensin receptor blocker, a calcium channel blocker, a nitrate or magnesium chloride.

In some embodiments, the adjuvant is magnesium chloride and is present in the pharmaceutical composition, e.g., at a concentration ranging from about 0.1% (w/v) to about 50% (w/v), from about 0.1% (w/v) to about 30% (w/v), from about 5% (w/v) to about 30% (w/v), from about 1% (w/v) to about 25% (w/v), from about 15% (w/v) to about 25% (w/v), from about 0.5% (w/v) to about 5% (w/v), from about 0.5% (w/v) to about 1% (w/v), from about 0.5% (w/v) to about 1.5% (w/v), from 1.5% (w/v) to 3.5% (w/v), from about 0.5% (w/v) to about 3.5% (w/v), from about 0.5% (w/v) to about 3.0% (w/v), from about 2.5% (w/v) to about 3% (w/v), from about 2.0% (w/v) to about 4% (w/v), from about 2.0% (w/v) to about 3.0% (w/v), from about 4.5% (w/v) to about 5% (w/v), about 0.9% (w/v), about 1% (w/v), about 2% (w/v), about 2.8% (w/v), about 3% (w/v), about 4.7% (w/v), about 5% (w/v), about 10% (w/v), about 15% (w/v), or about 20% (w/v) in the pharmaceutical composition.

In certain embodiments, the adjuvant is magnesium chloride and is present in any of the pharmaceutical compositions described herein at a concentration ranging from about 0.1% (w/v) to about 10% (w/v), from about 0.2% (w/v) to about 5% (w/v), or from about 0.3% (w/v) to about 3% (w/v), or any single concentration value or sub-range therein.

The ACE inhibitor may comprise, e.g., enalapril, captopril, lisinopril, benazepril, enalaprilat, espirapril, fosinopril, moexipril, quinapril, ramipril, perindopril, trandolapril, pharmaceutically acceptable salts thereof and combinations thereof. The angiotensin receptor blocker may be selected from, e.g., valsartan, losartan, irbesartan, telmisartan, eprosartan, candesartan, olmesartan, saprisartan, tasosartan, elisartan, pharmaceutically acceptable salts thereof and combinations thereof. The calcium channel blocker may be selected from, e.g., amlodipine, anipamil, barnidipine, benidipine, bepridil, darodipine, diltiazem, efonidipine, felodipine, isradipine, lacidipine, lercanidipine, lidoflazine, manidipine, mepirodipine, nicardipine, nifedipine, niludipine, nilvadipine, nimodipine, nisoldipine, nitrendipine, perhexiline, tiapamil, verapamil, pharmaceutically acceptable salts thereof or combinations thereof.

In certain embodiments, the adjuvant comprises a nitric oxide inducer. The nitric oxide inducer can be, e.g., an amino acid (e.g., arginine). The nitric oxide inducer can be, without limitation, selected from the group consisting of L-arginine, L-homoarginine, N-hydroxy-L-arginine, nitrosated analogs thereof, nitrosylated analogs thereof, precursors thereof and combinations thereof. The nitrosated analogs may be selected from, e.g., the group consisting of nitrosated L-arginine, nitrosated N-hydroxy-L-arginine, nitrosated L-homoarginine and combinations thereof. The nitrosylated analogs may be selected from, e.g., the group consisting of nitrosylated L-arginine, nitrosylated N-hydroxy-L-arginine, nitrosylated L-homoarginine and combinations thereof. Also, the precursor may be selected from, e.g., the group consisting of citrulline, ornithine, glutamine, lysine and combinations thereof In one embodiment, the adjuvant is L-arginine and is present in the pharmaceutical composition, e.g., at a concentration ranging from about 0.1% to about 50%, from about 5% to about 30%, from about 15% to about 25%, or about 20% (w/v) of L-arginine per pharmaceutical composition.

In certain embodiments, the nitric oxide inducer comprises arginase inhibitors, substrates for nitric oxide synthase, nitroglycerin, amyl nitrate, and combinations thereof. In certain embodiments, the arginase inhibitor may be, e.g., selected from the group consisting of N-hydroxy-L-arginine, 2(S)-amino-6-boronohexanoic or combinations thereof. In other embodiments, the substrate for nitric oxide synthase is selected from the group consisting of cytokines, adenosine, bradykinin, calreticulin, bisacodyl, phenolphthalein, and combinations thereof.

In other embodiments, the adjuvant comprises niacin, a niacin derivative, a niacin metabolite, or a combination thereof. The niacin derivative may be, e.g., selected from the group consisting of acifran, acipimox, niceritrol, isonicotinic acid, isonicotinohydrazide, pyridine carboxylic acid derivatives, 3-pyridine acetic acid, 5-methylnicotinic acid, pyridazine-4-carboxylic acid, pyrazine-2-carboxylic acid, and combinations thereof. In certain embodiments, the niacin derivative is an ester of nicotinic acid, e.g., an alkyl ester of nicotinic acid such as methyl nicotinate. In other embodiments, the niacin metabolite may be, e.g., selected from the group consisting of nicotinuric acid, nicotinamide, 6-hydroxy nicotinamide, N-methylnicotinamide, nicotinamide-N-oxide, N-methyl-2-pyridone-5-carboxamide, N-methyl-4-pyridone-5-carboxamide, and combinations thereof. In certain embodiments, the adjuvant is niacin and is present in the pharmaceutical composition at a concentration ranging from about 0.1% to about 15%, from about 0.5% to about 5%, about 1%, about 2%, or about 3% (w/v) of niacin per pharmaceutical composition.

In certain embodiments, the adjuvant comprises a local anesthetic. The local anesthetic may be, e.g., an ester-based local anesthetic or an amide-based local anesthetic. The ester-based local anesthetic may be, e.g., selected from the group consisting of benzocaine, chloroprocaine, proparacaine, tetracaine, and combinations thereof. Also, the amide-based local anesthetic may be, e.g., selected from the group consisting of articaine, bupivacaine, dibucaine, lidocaine, mepivacaine, prilocaine, ropivacaine, and combinations thereof.

In certain embodiments, the adjuvant comprises a phosphodiesterase inhibitor. The phosphodiesterase inhibitor may be, e.g., selected from the group consisting of phosphodiesterase 1 inhibitors, phosphodiesterase 2 inhibitors, phosphodiesterase 3 inhibitors, phosphodiesterase 4 inhibitors, phosphodiesterase 5 inhibitors and combinations thereof. In other embodiments, the phosphodiesterase inhibitor may be, e.g., selected from the group consisting of vinpocetine, EHNA (erythro-9-(2-hydroxy-3-nonyl)adenine), anagrelide, enoximine, cilomilast, etazolate, glaucine, ibudilast, mesembrine, rolipram, pentoxifylline, piclamilast, dipyridamole, acetildenafil, avanafil, sildenafil, tadalafil, udenafil, vardenafil, milrinone, amrinone and combinations thereof.

In certain embodiments, the pharmaceutical composition and dosage forms disclosed herein comprise from about 0.1%, about 0.2%, about 0.3%, about 0.4%, about 0.5%, about 0.6%, about 0.7%, about 0.8%, about 0.9%, or about 1% to about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 60%, about 70%, or about 80% (w/v) of an adjuvant per dosage form. In certain embodiments, the pharmaceutical composition and dosage forms disclosed herein comprises from about 0.1% to about 30%, from about 0.5% to about 25%, or from about 1% to about 20% (w/v) of an adjuvant per dosage form.

### Active Agents

The delivery systems and pharmaceutical compositions disclosed herein include various active agents or their pharmaceutically acceptable salts. Pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like, and metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like.

The delivery systems and pharmaceutical compositions disclosed herein include an emergency rescue active pharmaceutical ingredient (ER-API). In certain embodiments, the ER-API is epinephrine, atropine, or a pharmaceutically acceptable salt thereof, or a combination thereof.

In certain embodiments, the ER-API is epinephrine or a pharmaceutically acceptable salt thereof which is present in a pharmaceutical formulation at about 0.001 mg/ml to about 10 mg/ml, about 0.005 mg/ml to about 9 mg/ml, about 0.01 mg/ml to about 8 mg/ml, about 0.05 mg/ml to about 7 mg/ml, about 0.1 mg/ml to about 5 mg/ml, about 0.3 mg/ml to about 2.5 mg/ml, about 0.5 mg/ml to about 1.5 mg/ml, or about 1 mg/ml, and is adapted for parenteral administration.

In certain embodiments, the ER-API is epinephrine or a pharmaceutically acceptable salt thereof and the pharmaceutical formulation (e.g., parenteral formulation) may comprise a dose (of epinephrine base) from any of about 0.05 mg, about 0.1 mg, about 0.15 mg, or about 0.2 mg, to any of about 0.25 mg, about 0.3 mg, about 0.35 mg, about 0.4 mg, about 0.45 mg, or about 0.5 mg.

In certain embodiments, the pharmaceutical composition is a parenteral formulation comprising a therapeutically effective amount of epinephrine or a pharmaceutically acceptable salt thereof and a parenterally acceptable adsorption enhancing amount of magnesium chloride, wherein the parenterally acceptable adsorption enhancing amount of magnesium chloride ranges from about 0.1% (w/v) to about 10% (w/v), from about 0.2% (w/v) to about 5% (w/v), or from about 0.3% (w/v) to about 3% (w/v), or any single concentration value or sub-range therein.

In certain embodiments, the ER-API is atropine or a pharmaceutically acceptable salt thereof which is present in a pharmaceutical formulation at about 0.001 mg/ml to about 10 mg/ml, about 0.005 mg/ml to about 9 mg/ml, about 0.01 mg/ml to about 8 mg/ml, about 0.05 mg/ml to about 7 mg/ml, about 0.1 mg/ml to about 5 mg/ml, about 0.3 mg/ml to about 2.5 mg/ml, about 0.5 mg/ml to about 1.5 mg/ml, or about 1 mg/ml, and is adapted for parenteral administration.

In certain embodiments, the ER-API is atropine or a pharmaceutically acceptable salt thereof and the pharmaceutical formulation (e.g., parenteral formulation) may comprise a dose (of atropine base) from any of about 0.05 mg, about 0.1 mg, about 0.15 mg, or about 0.2 mg, about 0.25 mg, about 0.3 mg, about 0.35 mg, about 0.4 mg, about 0.45 mg, or about 0.5 mg to any of about 1 mg, about 1.25 mg, about 1.5 mg, about 1.75 mg, about 2 mg, about 2.25 mg, about 2.5 mg, about 2.75 mg, about 3 mg, about 3.5 mg, about 4 mg, about 4.5 mg, or about 5 mg.

In certain embodiments, the pharmaceutical composition is a parenteral formulation comprising a therapeutically effective amount of atropine or a pharmaceutically acceptable salt thereof and a parenterally acceptable adsorption enhancing amount of magnesium chloride, wherein the parenterally acceptable adsorption enhancing amount of magnesium chloride ranges from about 0.1% (w/v) to about 10% (w/v), from about 0.2% (w/v) to about 5% (w/v), or from about 0.3% (w/v) to about 3% (w/v), or any single concentration value or sub-range therein.

### Prophylactic Treatment

It is an object of certain embodiments of the present invention to provide a method to prevent or minimize an allergic reaction or poisoning in a subject that is at risk for exposure to an allergen or poison. For example, law enforcement personnel or first medical responders can be pre-treated with an ER-API according to the present invention prior to entering an environment or locale (e.g., a crime scene or emergency situation) where they suspect that poison (e.g., nerve agent) may have been intentionally or unintentionally released, or are otherwise present. Also, workers at environmental disaster areas involving allergens or poisons may be pretreated to avoid toxicity of allergens or poisons that may be present in the environment. In the embodiments directed to methods of prophylactic treatment, the administered compositions can include, but not be limited to the pharmaceutical compositions as disclosed herein. For example, the administration of an ER-API for a prophylactic treatment can utilize the presently disclosed formulations for intramuscular or subcutaneous administration or can utilize oral, nasal, pulmonary, transdermal, rectal, or intravenous routes of administering ER-API.

### Pharmaceutically Acceptable Excipients

The pharmaceutical compositions according to the present invention may comprise one or more pharmaceutically acceptable carriers and excipients appropriate for intramuscular or subcutaneous administration. Examples of possible pharmaceutically acceptable carriers and excipients are described in the Handbook of Pharmaceutical Excipients, American Pharmaceutical Association (6^{th} Edition, 2009 Publication), which is incorporated by reference herein. Carriers and excipients suitable for intramuscular and subcutaneous formulations include, but are not limited to, antioxidants, buffering agents, diluents, surfactants, solubilizers, stabilizers, hydrophilic polymers, additional absorption or permeability enhancers, preservatives, osmotic agents, isotonicity agents, pH adjusting agents, solvents, co-solvents, viscosity agents, gelling agents, suspending agents or combinations thereof.

Suitable surfactants for the formulations disclosed herein include, but are not limited to Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and the like, and combinations thereof.

Suitable isotonicity agents for the pharmaceutical compositions disclosed herein include, but are not limited to dextrose, lactose, sodium chloride, calcium chloride, magnesium chloride, sorbitol, sucrose, mannitol, trehalose, raffinose, polyethylene glycol, hydroxyethyl starch, glycine, and the like, and combinations thereof.

Suitable suspending agents for the formulations disclosed herein include, but are not limited to microcrystalline cellulose, carboxymethylcellulose sodium NF, polyacrylic acid, magnesium aluminum silicate, xanthan gum, and the like, and mixtures thereof. In certain embodiments, the pharmaceutical compositions may include one or more suspending agents in an amount of from about 0.1 wt % to about 15 wt %, or from about 0.25 wt % to about 10 wt %, or from about 1 wt % to about 8 wt %, of the total weight of the pharmaceutical composition.

### Method of Providing Emergency Rescue

In certain embodiments, the present disclosure is directed to a method of providing emergency rescue to a subject in need thereof. The method comprises administering to a subject in need thereof an ER-API, and optionally an adjuvant, such that the onset of action of the ER-API is achieved in sufficient time to reverse or partially reverse the allergic reaction or poisoning. In certain embodiments, the present invention is intended to be urgently administered to a subject experiencing a medical emergency precipitated by an allergic reaction (e.g., anaphylactic shock) or poison. In such circumstances, the pharmaceutical composition will typically be administered by a medical practitioner, emergency medical technician, law enforcement member, family member, acquaintance, or bystander upon observing the subject experiencing the symptoms of an allergic reaction or poisoning. In some embodiments, the method further comprises, before the administering step, identifying that the subject is experiencing an allergic reaction or poisoning.

The allergic reaction or poisoning treated by the present invention can result from any allergen or poison currently known or those that would be readily appreciated by an ordinary skilled artisan (in formulation and medical fields) for such use, including but not limited to any of the following: chemical nerve agents, insecticide poisoning, allergens (e.g., pollen, nuts such as peanuts and tree nuts, milk, eggs, shellfish, wheat, soy, fish, insect bites, bee stings, and the like), or combinations thereof.

In some embodiments, the ER-API and the adjuvant are each administered separately. In other embodiments, the ER-API and the adjuvant are administered together as a combination in a single dosage form. In some embodiments, the ER-API and the adjuvant are both administered via the same route of administration, i.e., intramuscular or subcutaneous. In other embodiments, the ER-API and the adjuvant are administered via different routes of administration.

In one embodiment, the ER-API and the adjuvant are both administered to a subject in need thereof via intramuscular administration.

In another embodiment, the ER-API and the adjuvant are administered to a subject in need thereof via subcutaneous administration.

### Intranasal Formulations

In certain embodiments, the present invention is directed to a pharmaceutical formulation suitable for administration intranasally or via inhalation. The intranasal pharmaceutical formulation may comprise a therapeutically effective amount of an ER-API (e.g., epinephrine or atropine or pharmaceutically acceptable salts thereof) and a pharmaceutically acceptable adjuvant (e.g., an intranasally acceptable adjuvant) that promotes the absorption rate of the ER-API post intranasal or inhalative administration. The adjuvant may include any of the adjuvants described hereinbefore, such as magnesium chloride.

In certain embodiments, the invention is directed to a pharmaceutical composition comprising a therapeutically effective amount of an ER-API and an intranasally acceptable adjuvant, wherein the formulation provides a time to onset of action (i.e., the first detectable therapeutic effect associated with administration of an ER-API, e.g., detectable lessening or reduction of any of the symptoms associated with allergic reaction or poisoning) that is shorter than the time to onset of action of a comparative pharmaceutical composition without the intranasally acceptable adjuvant.

In other embodiments, the invention is directed to a pharmaceutical composition comprising a therapeutically effective amount of ER-API and an intranasally acceptable adjuvant, wherein the formulation provides a mean time to maximum plasma concentration that is shorter than the mean time to maximum plasma concentration of a comparative pharmaceutical composition without the intranasally acceptable adjuvant, post administration intranasally or via inhalation to a population of subjects (e.g., healthy or otherwise healthy subjects). For instance, the mean Tₘₐₓ of the present invention may be, e.g., about 1.1 times shorter, about 1.2 times shorter, about 1.3 times shorter, about 1.4 times shorter, about 1.5 times shorter, about 1.6 times shorter, about 1.7 times shorter, about 1.8 times shorter, about 1.9 times shorter, about 2 times shorter, about 3 times shorter or about 4 times shorter than that of a comparative formulation without an adjuvant.

In certain embodiments, the invention is directed to a pharmaceutical composition comprising a therapeutically effective amount of an ER-API and an intranasally acceptable adjuvant, wherein the formulation provides a mean maximum plasma concentration of the ER-API (Cₘₐₓ) that is greater than the mean maximum plasma concentration of ER-API of a comparative formulation without an adjuvant, post administration intranasally or via inhalation to a population of subjects (e.g., healthy or otherwise healthy subjects). For instance, Cₘₐₓ may be about 1.1 times greater, about 1.2 times greater, about 1.3 times greater, about 1.4 times greater, about 1.5 times greater, about 1.6 times greater, about 1.7 times greater, about 1.8 times greater, about 1.9 times greater, or about 2 times greater, about 4 times greater, about 8 times greater, about 12 times greater or about 16 times greater than that of a comparative formulation without an adjuvant.

In certain embodiments, the invention is directed to a pharmaceutical composition comprising a therapeutically effective amount of ER-API and an intranasally acceptable adjuvant, wherein the formulation provides AUC_{inf} that is greater than the AUC_{0-inf} of a comparative formulation without an adjuvant, post intranasal administration to a population of subjects (e.g., healthy or otherwise healthy subjects). For instance, AUC_{inf} may be about 1.1 times greater, about 1.2 times greater, about 1.3 times greater, about 1.4 times greater, about 1.5 times greater, about 1.6 times greater, about 1.7 times greater, about 1.8 times greater, about 1.9 times greater, or about 2 times greater, about 4 times greater, about 6 times greater, about 8 times greater or about 10 times greater than that of a comparative formulation without an adjuvant.

In certain embodiments, the invention is directed to a pharmaceutical composition comprising a therapeutically effective amount of ER-API and an intranasally acceptable adjuvant, wherein the formulation provides an AUC_{0.25}, AUC_{0.5}, AUC_{0.75}, or AUC₁ that is greater than the respective AUC_{0.25}, AUC_{0.5}, AUC_{0.75}, or AUC₁ of a comparative formulation without an adjuvant, post intranasal administration to a population of subjects (e.g., healthy or otherwise healthy subjects). For instance, AUC_{0.25}, AUC_{0.5}, AUC_{0.75}, or AUC₁ may be about 1.1 times greater, about 1.2 times greater, about 1.3 times greater, about 1.4 times greater, about 1.5 times greater, about 1.6 times greater, about 1.7 times greater, about 1.8 times greater, about 1.9 times greater, or about 2 times greater than the respective AUC_{0.25}, AUC_{0.5}, AUC_{0.75}, or AUC₁ of a comparative formulation without an adjuvant.

In certain embodiments, the mean time to maximum plasma concentration, the maximum plasma concentration, and/or the AUC_{inf} of the pharmaceutical composition for administration intranasally or via inhalation may fall within the same ranges as described hereinbefore for parenteral formulations.

In certain embodiments, the pharmacokinetic values described herein may be obtained from an individual subject (healthy or in therapeutic need thereof) or from a plurality of subjects (healthy or in therapeutic need thereof) post intranasal administration or post administration via inhalation of any of the pharmaceutical compositions disclosed herein that are suitable for intranasal administration or for administration via inhalation.

The role of the adjuvant is to promote or quicken the systemic absorption rate of the ER-API (e.g., epinephrine or pharmaceutically acceptable salt thereof or atropine or pharmaceutically acceptable salt thereof) post intranasal administration or post administration via inhalation. In certain embodiments, the adjuvant is magnesium chloride.

In some embodiments, the adjuvant is magnesium chloride and is present in the intranasal pharmaceutical composition, e.g., at a concentration ranging from about 0.1% (w/v) to about 50% (w/v), from about 0.1% (w/v) to about 30% (w/v), from about 5% (w/v) to about 30% (w/v), from about 1% (w/v) to about 25% (w/v), from about 15% (w/v) to about 25% (w/v), from about 0.5% (w/v) to about 5% (w/v), from about 0.5% (w/v) to about 1% (w/v), from about 0.5% (w/v) to about 1.5% (w/v), from about 1.5% (w/v) to about 2.5% (w/v), from about 0.5% (w/v) to about 3.5% (w/v), from about 0.5% (w/v) to about 3.0% (w/v), from about 2.5% (w/v) to about 3% (w/v), from about 2.0% (w/v) to about 4%(w/v), from about 2.0% (w/v) to about 3.0% (w/v), from about 4.5% (w/v) to about 5% (w/v), about 0.9% (w/v), about 1% (w/v), about 2% (w/v), about 2.8% (w/v), about 3% (w/v), about 4.7% (w/v), about 5% (w/v), about 10% (w/v), about 15% (w/v), or about 20% (w/v) in the pharmaceutical composition.

In certain embodiments, any of the intranasal or inhalative pharmaceutical compositions include magnesium chloride as the adjuvant at a concentration ranging from about 0.1% (w/v) to about 10% (w/v), from about 0.2% (w/v) to about 5% (w/v), or from about 0.3% (w/v) to about 3% (w/v), or any single concentration value or sub-range therein.

In certain embodiments, pharmaceutical compositions for administration intranasally or via inhalation may be an aqueous solution, suspension, or an emulsion. Such pharmaceutical compositions may be administered to a subject via a device adapted for nasal administration, such as, without limitations, nasal atomizer/nebulizer device, a metered multi-dose spray pump, single-dose or bi-dose spray devices.

In certain embodiments, the pharmaceutical formulation (e.g., formulation for inhalation) comprises a dose (of ER-API) from any of about 0.01 mg, about 0.05 mg, about 0.1 mg, about 0.125 mg, about 0.15 mg, about 0.175 or about 0.2 mg to any of about 0.225 mg, about 0.25 mg, about 0.275 mg, about 0.3 mg, about 0.35 mg, about 0.4 mg, about 0.45 mg, or about 0.5 mg per inhalation.

In certain embodiments, the pharmaceutical composition is a formulation suitable for intranasal administration or for inhalative administration and comprises a therapeutically effective amount of epinephrine or a pharmaceutically acceptable salt thereof and an adsorption enhancing amount of magnesium chloride, wherein the adsorption enhancing amount of magnesium chloride ranges from about 0.1% (w/v) to about 10% (w/v), from about 0.2% (w/v) to about 5% (w/v), or from about 0.3% (w/v) to about 3% (w/v), or any single concentration value or sub-range therein.

In certain embodiments, the pharmaceutical composition is a formulation suitable for intranasal administration or for inhalative administration and comprises a therapeutically effective amount of atropine or a pharmaceutically acceptable salt thereof and an adsorption enhancing amount of magnesium chloride, wherein the adsorption enhancing amount of magnesium chloride ranges from about 0.1% (w/v) to about 10% (w/v), from about 0.2% (w/v) to about 5% (w/v), or from about 0.3% (w/v) to about 3% (w/v), or any single concentration value or sub-range therein.

### Drug Delivery Systems and Kits

In certain embodiments, the present invention is directed to a drug delivery system or to a kit containing an injection device and any of the pharmaceutical formulations (e.g., parenteral) disclosed herein. In certain embodiments, the injection device is pre-filled with the pharmaceutical formulation. In certain embodiments, the injection device is a syringe, a vial, an injection pen, or an autoinjector, which is pre-filled with the pharmaceutical formulation disclosed herein.

In certain embodiments, the present invention is directed to a drug delivery system or to a kit containing a device for intranasal administration or administration via inhalation (e.g., nasal atomizer/nebulizer device, a metered multi-dose spray pump, single-dose or bi-dose spray devices). In certain embodiments, the device for intranasal administration or administration via inhalation is pre-filled with the pharmaceutical formulation.

In certain embodiments, the drug delivery system or kit comprises an active agent and an adjuvant in separate containers (e.g., separate vials, separate syringe barrels, separate compartments, and the like). In one embodiment, the ER-API (e.g., epinephrine or a pharmaceutically acceptable salt thereof, or atropine or a pharmaceutically acceptable salt thereof) is in one container and an adjuvant (e.g., MgCl₂) is in another container; and the ER-API is mixed with the adjuvant prior to administration (e.g., parenterally, intranasally, or via inhalation).

In certain embodiments, the active agent (e.g., ER-API such as epinephrine or a pharmaceutically acceptable salt thereof, or atropine or a pharmaceutically acceptable salt thereof) is in solution or in powder form in the drug delivery system or kit. In certain embodiments, the adjuvant is in solution or in powder form in the drug delivery system or kit.

In one embodiment, the drug delivery system or kit comprises a solution of an active agent (e.g., ER-API such as epinephrine or a pharmaceutically acceptable salt thereof, or atropine or a pharmaceutically acceptable salt thereof) in one container and an adjuvant (e.g., MgCl₂) solution in another container. The active agent solution and adjuvant solution are mixed prior to administration. In one embodiment, the active agent solution is in one compartment of an auto-injector (or device for administration intranasally or via inhalation) and the adjuvant solution is in another compartment in an auto-injector (or device for administration intranasally or via inhalation) and the two solutions are mixed in the auto-injector prior to administration (e.g., parenterally, intranasally, or via inhalation). In another embodiment, the active agent solution is in one vial, the adjuvant solution is in another vial, and the contents of the vials are mixed prior to administration (e.g, by transferring the content of one vial into another vial with a syringe and needle which could be part of the kit described herein).

In one embodiment, the drug delivery system or kit described herein comprises a solution of an active agent (e.g., ER-API such as epinephrine or a pharmaceutically acceptable salt thereof, or atropine or a pharmaceutically acceptable salt thereof) in one container and an adjuvant (e.g., MgCl₂) powder in another container. The active agent solution and adjuvant powder are mixed prior to administration. In one embodiment, the active agent solution is in one compartment of an auto-injector (or a device for administration intranasally or via inhalation) and the adjuvant powder is in another compartment in an auto-injector (or a device for administration intranasally or via inhalation) and the powder and solution are mixed in the auto-injector (or a device for administration intranasally or via inhalation) prior to administration. In another embodiment, the active agent solution is in one vial (or pre-filled syringe barrel or the like), the adjuvant powder is in another vial, and the active agent solution may be added to the adjuvant powder (e.g., by transferring the active agent solution into the adjuvant powder container with a syringe and needle which could be part of the kit described herein) to suspend or dissolve the adjuvant powder prior to administration (e.g., parenterally, intranasally, or via inhalation).

In one embodiment, the drug delivery system or kit described herein comprises a powder of an active agent (e.g., ER-API such as epinephrine or a pharmaceutically acceptable salt thereof, or atropine or a pharmaceutically acceptable salt thereof) in one container and an adjuvant (e.g., MgCl₂) solution in another container. The active agent powder and adjuvant solution are mixed prior to administration (e.g., parenterally, intranasally, or via inhalation). In one embodiment, the active agent powder is in one compartment of an auto-injector (or a device for administration intranasally or via inhalation) and the adjuvant solution is in another compartment of an auto-injector (or a device for administration intranasally or via inhalation) and the powder and solution are mixed in the auto-injector (or a device for administration intranasally or via inhalation) prior to administration. In another embodiment, the active agent powder is in one vial, the adjuvant solution is in another vial (or pre-filled syringe barrel or the like), and the adjuvant solution is added to the active agent powder (e.g., by transferring the adjuvant solution into the active agent powder container with a syringe and needle which could be part of the kit described herein) to suspend or dissolve the active agent powder prior to administration (e.g., parenterally, intranasally, or via inhalation).

In one embodiment, the drug delivery system or kit described herein comprises a powder of an active agent (e.g., ER-API such as epinephrine or a pharmaceutically acceptable salt thereof or atropine or a pharmaceutically acceptable salt thereof) in one container, an adjuvant (e.g., MgCl₂) powder in another container, and a solvent in yet another container. The active agent powder, adjuvant powder, and solvent are mixed prior to administration. In one embodiment, the active agent powder is in one compartment of an auto-injector (or device for administration intranasally or via inhalation), the adjuvant powder is in another compartment of an auto-injector (or device for administration intranasally or via inhalation), and a solvent is in yet another compartment of an auto injector such that the powders are suspended or dissolved in the solvent prior to administration (e.g., parenterally, intranasally, or via inhalation). In another embodiment, the active agent powder is in one vial, the adjuvant powder is in another vial, and the solvent is in yet another vial (or pre-filled syringe barrel or the like), and the solvent is added to the active agent powder and/or to the adjuvant powder (e.g., by transferring the solvent into the active agent powder and/or the adjuvant powder container(s) with a syringe and needle which could be part of the kit described herein) to suspend or dissolve the powders prior to administration (e.g., parenterally, intranasally, or via inhalation).

In certain embodiments, the drug delivery system or kit described herein comprises an active agent (e.g., ER-API such as epinephrine or a pharmaceutically acceptable salt thereof, or atropine or a pharmaceutically acceptable salt thereof) and an adjuvant (e.g., MgCl₂) combined together in a powder form in one container, and a pharmaceutically acceptable solvent is stored in another container, prior to administration (e.g., parenterally, intranasally, or via inhalation). In one embodiment, the active agent powder and adjuvant powder are mixed together in one compartment of an auto-injector (or device for administration intranasally or via inhalation) and a pharmaceutically acceptable solvent may be stored in another compartment in an auto injector (or device for administration intranasally or via inhalation) such that the powder mixture is suspended or dissolved in the solvent prior to administration. In another embodiment, the active agent powder and the adjuvant powder are mixed together in one vial, and the solvent may be in another vial (or pre-filled syringe barrel or the like), and the solvent is added to the powder mixture (e.g., by transferring the solvent into the powder mixture container with a syringe and needle which could be part of the kit described herein) to suspend or dissolve the powder mixture prior to administration (e.g., parenterally, intranasally, or via inhalation).

In certain embodiments, the drug delivery system or kit described herein comprises a needle having a needle gauge ranging from about 18-gauge to about 35-gauge. For instance, the needle used for parenteral administration of any of the pharmaceutical compositions described herein (whether it is a separate needle or a needle of an autoinjector device) may be 18-gauge, 20-gauge, 21-gauge, 22-gauge, 23-gauge, 25-gauge, 27-gauge, 29-gauge, 31-gauge, or 33-gauge.

Concentration ranges and/or values of active agents and/or adjuvants expressed in % (w/v) disclosed previously refer to the final concentrations when all components are mixed together just prior to administration.

### EXAMPLES

The following examples are set forth to assist in understanding the invention and should not be construed as specifically limiting the invention described and claimed herein. Such variations of the invention, including the substitution of any or all equivalents now known or later developed, which would be within the purview of those skilled in the art, and changes in formulation or minor changes in therapeutic design, are to be considered to fall within the scope of the invention incorporated herein.

### Example 1

### Epinephrine Study Design

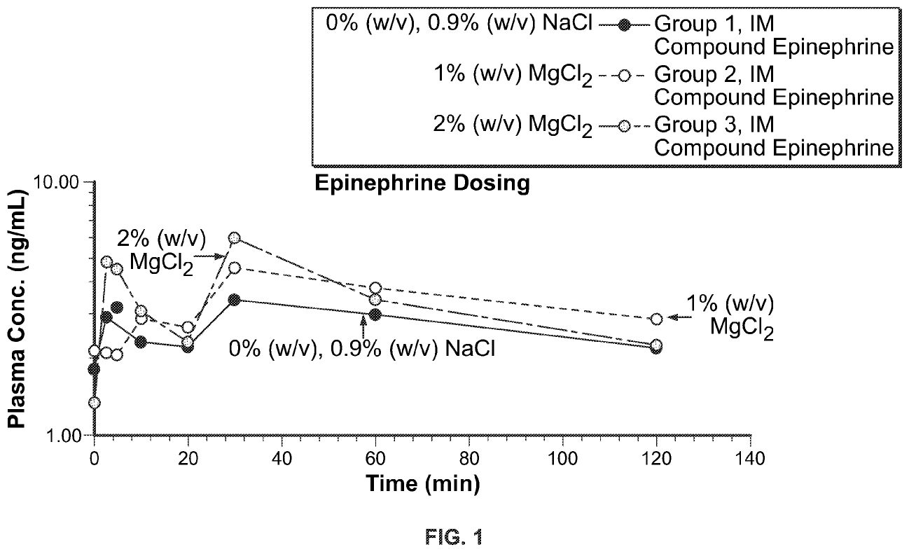

Absorption of Epinephrine (0.2 mg dose, 1 mg/mL concentration) with varying concentrations of MgCl₂ adjuvant (0% MgCl₂ in the saline samples, 1% MgCl₂ in water, and 2% MgCl₂ in water) was tested. Each Epinephrine formulation was administered intramuscularly to three dog subjects. Eight blood samples at varying time points (pre-dose, 2.5 minutes post dose, 5 minutes post dose, 10 minutes post dose, 20 minutes post dose, 30 minutes post dose, 60 minutes post dose, and 120 minutes post dose) were taken from each subject to evaluate the pharmacokinetic profile of Epinephrine absorption. Details of the Epinephrine study design are summarized in Table 1 below with reference to Groups 1-3. The plasma concentration of Epinephrine after intramuscular administration in dogs with varying concentration of MgCl₂ is summarized in FIG. 1 and in Table 2 below.

TABLE 1
Epinephrine Study Design

| | | | | | Dosing | Dose | | Blood |
|---|---|---|---|---|---|---|---|---|
| | Test | Dosing | | Dose | Concentration | Volume | | Sampling |
| | Article | Route | N= | (mg) | (mg/mL) | (mL) | Vehicle | Points |
| 1 | Epinephrine | IM | 3 | 0.2 | 1 | 0.2 | 0.9% | Pre-dose, |
| | | | | | | | NaCl | 2.5 minutes, |
| | | | | | | | (saline) | 5 minutes, |
| 2 | Epinephrine | IM | 3 | 0.2 | 1 | 0.2 | 1% | 10 minutes, |
| | | | | | | | MgCl₂ | 20 minutes, |
| | | | | | | | in water | 30 minutes, |
| 3 | Epinephrine | IM | 3 | 0.2 | 1 | 0.2 | 2% | 60 minutes, and |
| | | | | | | | MgCl₂ | 120 minutes |
| | | | | | | | in water | post dose |

TABLE 2
Epinephrine Pharmacokinetic Data Summary

| | Group 1 | Group 2 | Group 3 |
|---|---|---|---|
| | IM | IM | IM |
| | Epinephrine | Epinephrine | Epinephrine |
| Parameters | 0.9% NaCl | 1% MgCl₂ | 2% MgCl₂ |
| Dose (mg) | 0.2 | 0.2 | 0.2 |
| Cₘₐₓ (ng/mL) | 3.65 | 4.58 | 5.99 |
| Tₘₐₓ (hr) | 0.528 | 0.667 | 0.347 |
| T_{1/2} (hr) | ND* | ND* | ND* |
| MRT₂ (hr) | 0.948 | 0.981 | 0.895 |
| AUC₂(hr · ng/mL) | 5.44 | 6.80 | 6.58 |
| AUC_{∞} | ND* | ND* | ND* |
| (hr · ng/mL) | | | |

*ND—not determined

As seen in FIG. 1 and in Table 2, Epinephrine formulations with MgCl₂ (groups 2 and 3) achieved a higher Cₘₐₓ than Epinephrine formulations without MgCl₂. It can also be observed from FIG. 1 and Table 2 that Epinephrine formulations with 2% MgCl₂ shortened the Tₘₐₓ as compared to Epinephrine formulations without MgCl₂. Additionally, the AUCₗₐₛₜ observed in Epinephrine formulations with MgCl₂ is greater than that of Epinephrine formulations without MgCl₂. Without being construed as limiting, it is believed, based on the above data, that the MgCl₂ enhances absorption of Epinephrine by increasing the Cₘₐₓ and/or decreasing the Tₘₐₓ and/or increasing the AUCₗₐₛₜ.

### Example 2

### Atropine Study Design

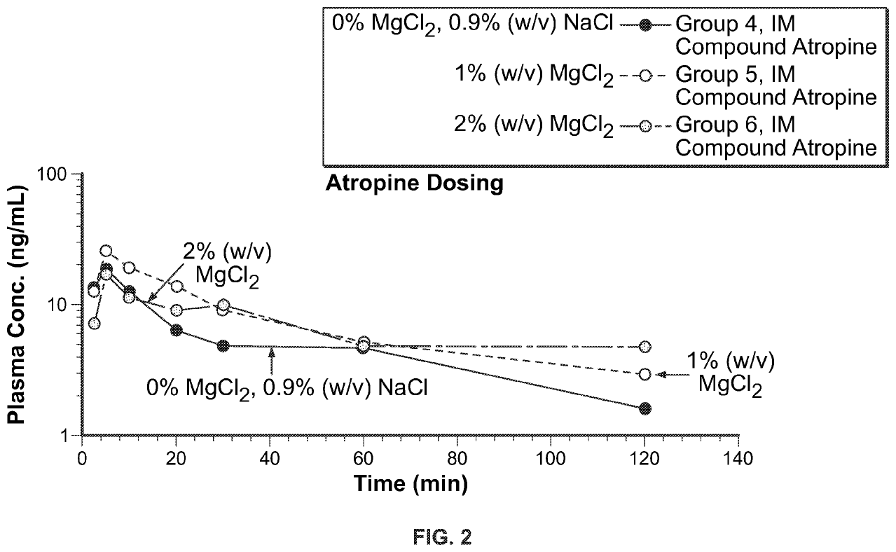

Absorption of Atropine (0.2 mg dose, 1 mg/mL concentration) with varying concentrations of MgCl₂ adjuvant (0% MgCl₂ in the saline samples, 1% MgCl₂ in water, and 2% MgCl₂ in water) was tested. Each Atropine formulation was administered intramuscularly to three dog subjects. Eight blood samples at varying time points (pre-dose, 2.5 minutes post dose, 5 minutes post dose, 10 minutes post dose, 20 minutes post dose, 30 minutes post dose, 60 minutes post dose, and 120 minutes post dose) were taken from each subject to evaluate the pharmacokinetic profile of Atropine absorption. Details of the Atropine study design are summarized in Table 3 below with reference to Groups 4-6. The plasma concentration of Atropine after intramuscular administration in dogs with varying concentration of MgCl₂ is summarized in FIG. 2 and in Table 4 below.

TABLE 3
Study Design

| | | | | | Dosing | Dose | | Blood |
|---|---|---|---|---|---|---|---|---|
| | Test | Dosing | | Dose | Concentration | Volume | | Sampling |
| | Article | Route | N= | (mg) | (mg/mL) | (mL) | Vehicle | Points |
| 4 | Atropine | IM | 3 | 0.2 | 1 | 0.2 | 0.9% | Pre-dose, |
| | | | | | | | NaCl | 2.5 minutes, |
| | | | | | | | (saline) | 5 minutes, |
| 5 | Atropine | IM | 3 | 0.2 | 1 | 0.2 | 1% | 10 minutes, |
| | | | | | | | MgCl₂ in | 20 minutes, |
| | | | | | | | water | 30 minutes, |
| 6 | Atropine | IM | 3 | 0.2 | 1 | 0.2 | 2% | 60 minutes, and |
| | | | | | | | MgCl₂ in | 120 minutes |
| | | | | | | | water | post dose |

TABLE 4
Atropine Pharmacokinetic Data Summary

| | Group 4 | Group 5 | Group 6 |
|---|---|---|---|
| | IM | IM | IM |
| Test | Atropine | Atropine | Atropine |
| Article | 0.9% NaCl | 1% MgCl₂ | 2% MgCl₂ |
| Dose (mg) | 0.2 | 0.2 | 0.2 |
| Cₘₐₓ | 18.7 | 25.9 | 17.2 |
| (ng/mL) | | | |
| Tₘₐₓ (hr) | 0.0833 | 0.139 | 0.111 |
| T_{1/2} (hr) | ND* | ND* | ND* |
| MRT₂ (hr) | 0.558 | 0.628 | 0.785 |
| AUC₂ | 9.34 | 15.2 | 13.6 |
| (hr · ng/mL) | | | |
| AUC_{∞} | 13.9 | 19.3 | ND* |
| (hr · ng/mL) | | | |

*ND—not determined

As seen in FIG. 2 and in Table 4, Atropine formulations with 1% MgCl₂ (group 5) achieves a higher Cₘₐₓ than Atropine formulations without MgCl₂ (group 4). Additionally, the AUCₗₐₛₜ observed in Atropine formulations with MgCl₂ (groups 5 and 6) is greater than that of Atropine formulations without MgCl₂ (group 4). Without being construed as limiting, it is believed, based on the above data, that the MgCl₂ enhances absorption of Atropine by increasing the Cₘₐₓ and/or decreasing the Tₘₐₓ and/or increasing the AUCₗₐₛₜ.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is simply intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A parenteral formulation comprising a therapeutically effective amount of an emergency rescue active pharmaceutical ingredient (ER-API) and a parenterally acceptable absorption enhancing amount of adjuvant, wherein ER-API is not an opioid antagonist.

2. The parenteral formulation of claim 1, wherein the formulation promotes the systemic absorption rate of the ER-API upon injection as compared to the same formulation without the adjuvant.

3. The parenteral formulation of claim 1, wherein the formulation provides a shorter time to onset of emergency rescue action upon intramuscular or subcutaneous injection to a subject experiencing an allergic reaction or poisoning as compared to the same formulation without the adjuvant.

4. The parenteral formulation of claim 1, wherein the ER-API is epinephrine or a pharmaceutically acceptable salt thereof.

5. The parenteral formulation of claim 1, wherein the ER-API is atropine or a pharmaceutically acceptable salt thereof.

6. The parenteral formulation of claim 1, wherein the formulation provides a time to onset of emergency rescue action of about 5 minutes or less, about 4 minutes or less, about 3 minutes or less, about 2 minutes or less, and about 1 minute or less, post intramuscular or subcutaneous injection to a subject experiencing an allergic reaction or poisoning.

7. The parenteral formulation of claim 1, wherein the formulation provides a mean time to maximum plasma concentration of the ER-API of about 2.0 hours or less post intramuscular or subcutaneous administration to a population of healthy subjects.

8. The parenteral formulation of claim 1, wherein the formulation provides a mean time to maximum plasma concentration of the ER-API of about 1.5 hours or less, about 1 hour or less, about 0.75 hour or less, about 0.5 hour or less, about 20 minutes or less, about 15 minutes or less, about 10 minutes or less, about 5 minutes or less, about 3 minutes or less or about 1 minute or less post intramuscular or subcutaneous injection to a population of healthy subjects.

9. The parenteral formulation of claim 1, wherein the adjuvant comprises magnesium chloride.

10. The parenteral formulation of claim 1, wherein the adjuvant increases the rate of systemic absorption of the ER-API upon intramuscular or subcutaneous injection.

11. The parenteral formulation of claim 1, wherein the adjuvant is present in the parenteral formulation from about 0.1% (w/v) to about 50% (w/v).

12. A method of providing emergency rescue to a subject experiencing an allergic reaction or poisoning, comprising intramuscularly or subcutaneously administering to a subject in need thereof a formulation of claim 1.

13. A drug delivery system comprising an injection device containing a formulation of claim 1.

14. A kit comprising a therapeutically effective amount of an emergency rescue active pharmaceutical ingredient (ER-API) and an absorption enhancing amount of a parenterally acceptable adjuvant, wherein the ER-API and the adjuvant are independently in a solution form or in a powder form, and wherein the ER-API and the adjuvant are stored either in separate containers or in one container.

15. The kit of claim 14, wherein the ER-API is epinephrine or pharmaceutically acceptable salt thereof.

16. The kit of claim 14, wherein the ER-API is atropine or a pharmaceutically acceptable salt thereof.

17. The kit of claim 14, wherein the adjuvant is magnesium chloride.

18. The kit of claim 14, wherein the injection device is an auto-injector.

19. The kit of claim 18, wherein the auto-injector is pre-filled with a solution of the ER-API, a solution of the parenterally acceptable adjuvant, or both.

20. The kit of claim 14, further comprising a syringe and a needle.
